(19) United States
(12) Patent Application Publication
(10) Pub. No.: US 2025/0207578 A1
(43) Pub. Date: Jun. 26, 2025

(54) PERISTALTIC PUMP ACTUATED BY PIEZOELECTRIC TRANSDUCERS

(71) Applicant: Commissariat à l'Energie Atomique et aux Energies Alternatives, Paris (FR)

(72) Inventors: Jean-Pierre NIKOLOVSKI, Grenoble cedex 09 (FR); Elisabeth DELEVOYE, Grenoble cedex 09 (FR)

(73) Assignee: Commissariat à l'Energie Atomique et aux Energies Alternatives, Paris (FR)

(21) Appl. No.: 18/990,562

(22) Filed: Dec. 20, 2024

(30) Foreign Application Priority Data
Dec. 21, 2023 (FR) FR2314942

(51) Int. Cl. F04B 43/14; F04B 43/00; H10N 30/20; H10N 30/50

(52) U.S. Cl. CPC F04B 43/14; F04B 43/0081; H10N 30/2047; H10N 30/506

(57) ABSTRACT

Peristaltic pump, intended to pump a liquid along a capillary (2), the pumping resulting from a stress exerted on the capillary, successively along a pumping direction, the pump comprising:
K annular piezoelectric transducers (11ₖ), extending about a central axis (Δ),
K resonators (10ₖ), formed from a deformable solid material, each resonator being connected to a piezoelectric transducer, and extending about the central axis, becoming thinner towards the central axis, each resonator being configured to deform under the effect of the polarization of the piezoelectric transducer to which it is connected;
a control unit (20), configured to polarize each piezoelectric transducer; the pump being characterized in that:
each resonator is connected to a sleeve (3_{K}), extending about the central axis;
each resonator is arranged so that the sleeves of each resonator are aligned along a central axis.

### TECHNICAL FIELD

The technical field of the invention is a peristaltic pump configured for actuation by a piezoelectric transducer.

### PRIOR ART

Most pumps utilize movable parts, which can generate problems relating to reliability, wear and footprint. Peristaltic pumps are commonly used in the field of health. However, the repeated deformation of a hose, which causes the displacement of the liquid, can result in premature wear of the hose.

Patent application WO2013/41700 describes a non-peristaltic pump, which can be implantable, having piezoelectric actuation. A sleeve, arranged in the centre of a resonator, undergoes flexure under the effect of rotating deformation of the resonator, caused by piezoelectric transducers activated according to an ultrasonic frequency. The flexure of the sleeve generates a pumping effect, which results in the expulsion of the fluid. A reduction in the transverse cross-section of the resonator, in the vicinity of the sleeve or along said sleeve, makes it possible to amplify the vibrations propagating up to the sleeve. Such a pump is effective. However, it has been observed that the sleeve undergoes repeated flexure, which can result in wear. In addition, the pump is intended to be coupled to a fluid circuit. Control of the flow rate depends on the amplitude of vibration of the sleeve, which must be governed by the mechanical load of the fluid circuit, which is not easy.

In particular, the aim is for the pumping principle, the frequency and the amplitude of the ultrasonic vibration for pumping to be less dependent on the mechanical coupling between the pump and the fluid circuit.

Another aim is to design a peristaltic pump that makes it possible to perform pumping at a controlled flow rate with optimized energy expenditure, and which can be particularly compact.

### SUMMARY OF THE INVENTION

One object of the invention is a peristaltic pump, intended to pump a liquid along a capillary, the pumping resulting from a stress exerted on the capillary, successively along a pumping direction, the pump comprising:
K annular piezoelectric transducers, extending about a central axis, each piezoelectric transducer being configured to be polarized by a polarization electrode, K being an integer greater than or equal to 2;
K resonators, formed from a deformable solid material, each resonator being connected to a piezoelectric transducer, and extending about the central axis, becoming thinner towards the central axis, each resonator being configured to deform under the effect of the polarization of the piezoelectric transducer to which it is connected;
a control unit, configured to polarize each polarization electrode according to a polarization voltage, modulated according to a modulation frequency;
   the pump being characterized in that:
each resonator is connected to a sleeve, extending about the central axis;
each resonator is arranged so that the sleeves of each resonator are aligned along a central axis;
the control unit is configured to polarize each resonator in succession, as a function of their respective ranks, so that under the effect of the polarization, each resonator is deformed in succession, the deformation propagating up to the sleeve, and resulting in the successive deformation of the capillary arranged between the sleeves, about the central axis.

The stress can notably be a compression and/or a displacement along the central axis.

The modulation frequency can notably be greater than 20 kHz.

According to one option:
each resonator comprises a peripheral portion, delimited by two flat faces, and a central portion, extending between the peripheral portion and the sleeve, the central portion becoming thinner towards the sleeve;
each piezoelectric transducer extends along a flat face.

According to one option, the flat face along which each peripheral transducer extends is delimited by a shoulder, the piezoelectric transducer being force-fitted against the shoulder, so that the shoulder encircles the piezoelectric transducer.

According to one option, each resonator is coupled to at least one annular piezoelectric transducer, the annular piezoelectric transducer extending around the resonator, so as to cause a radial compression of the resonator, in the direction of the central axis.

Each piezoelectric transducer can be force-fitted around the resonator to which it is connected.

According to one option, each resonator is coupled to two annular piezoelectric transducers, extending around the resonator, and offset along a direction parallel to the central axis, the central unit being configured to power the piezoelectric transducers so as to cause a displacement of the sleeve along the central axis and/or a compression of the sleeve perpendicular to the central axis.

According to one option, each piezoelectric transducer having a dipole moment:
the dipole moments of the piezoelectric transducers coupled to the same resonator are oriented in the same direction, in which case the control unit is configured to power said piezoelectric transducers in phase;
the dipole moments of the piezoelectric transducers coupled to the same resonator are oriented in two opposite directions, in which case the control unit is configured to power said piezoelectric transducers in phase opposition;
so as to cause a radial compression of the sleeve that is predominant over a displacement of the sleeve along the central axis.

According to one option, each piezoelectric transducer having a dipole moment:
the dipole moments of the piezoelectric transducers coupled to the same resonator are oriented in the same direction, in which case the control unit is configured to power said piezoelectric transducers in phase opposition;
the dipole moments of the piezoelectric transducers coupled to the same resonator are oriented in two opposite directions, in which case the control unit is configured to power said piezoelectric transducers in phase;
so as to cause a displacement of the sleeve along the central axis that is predominant over a compression of the sleeve.

According to one option, the control unit is configured to power said piezoelectric transducers according to a predetermined phase shift, of between 0° and 180°, so as to obtain a predetermined ratio between the displacement of the sleeve along the central axis and the compression of the sleeve.

The pump can comprise the capillary, the capillary being for example force-fitted between each sleeve. The capillary can be rigid.

The invention will be more clearly understood on reading the description of the examples of embodiments given hereinafter, with reference to the figures listed below.

### FIGURES

FIGS. 1A and 1B show one embodiment of a peristaltic pump.

FIG. 2 shows a variant of the configuration described with reference to FIGS. 1A and 1B.

FIG. 3A shows another embodiment of a peristaltic pump.

FIG. 3B shows an example of an annular piezoelectric transducer.

FIG. 4 shows another embodiment of a peristaltic pump.

### DESCRIPTION OF PARTICULAR EMBODIMENTS

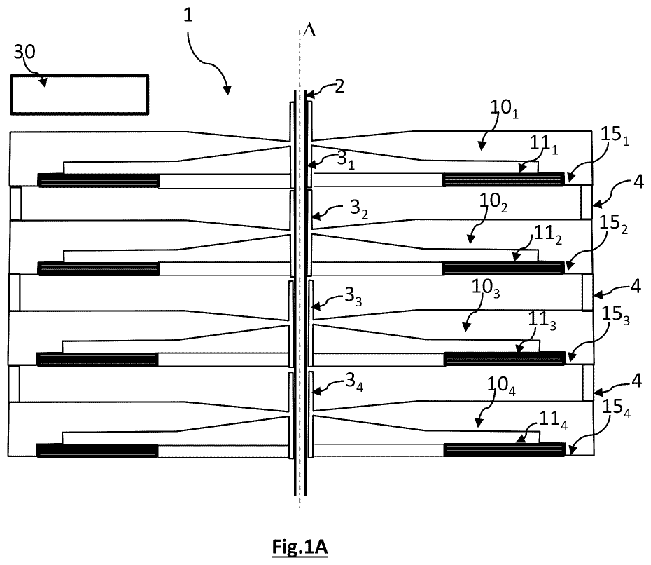

FIG. 1A shows a peristaltic pump, wherein a pumping effect is obtained by applying a progressive compression along a capillary 2. The capillary 2 is coaxial with a central axis.

The pump comprises K resonators 10ₖ, k being a rank assigned to each resonator. The minimum number of resonators is two, and the optimum number of resonators is between three and five. In the example shown, K=4. Four identical resonators 10₁, 10₂, 10₃, 10₄ are thus provided, aligned along the central axis. Each resonator extends in an annular shape, between a peripheral part comprising flat portions, having a constant thickness, and a part that becomes thinner towards the central axis. Each resonator becomes thinner, preferably progressively, up to a cylindrical sleeve 3ₖ that extends about the central axis. The progressive thinning makes it possible to increase the deformation forces transferred to the sleeve.

The capillary 2 is arranged along the central axis, between each sleeve. Preferably, the capillary 2 is rigid so that it can be force-fitted into the sleeves. Force-fitting minimizes the losses at the interfaces in the form of parasitic reflection of the mechanical energy or dissipation through friction. In addition, force-fitting does not require clamping means. The capillary can be formed from the same material as the resonator.

Each resonator comprises a shoulder 15ₖ, making it possible to grip the transducer to which it is coupled, so as to exert prestressing. Each shoulder encircles the piezoelectric transducer that it grips. Each piezoelectric transducer can thus be held without adhesive, through simple mechanical stress. This makes it possible to avoid the degradation over time of an adhesive due to ageing.

Each resonator 10ₖ is coupled to an annular piezoelectric transducer 11ₖ. FIG. 1B shows a detail of the mechanical coupling of an annular piezoelectric transducer 11ₖ connected to a resonator 10ₖ. The piezoelectric transducer 11ₖ comprises a layer of a piezoelectric material 13ₖ extending between at least one electrode 12ₖ and one counter electrode 14ₖ. When the resonator 10ₖ is made from an electrically conductive material, the electrode 12ₖ preferably has a slightly smaller outer diameter, for example between 0.2 mm and 0.5 mm, than the counter electrode 14ₖ so as to avoid an electrical short circuit between the electrodes 12ₖ and 14ₖ via the shoulder 15ₖ. The resonator 10ₖ is symmetrical relative to the central axis Δ. The resonator 10ₖ is annular about the central axis Δ. It becomes thinner towards the central axis. The thickness thereof, defined parallel to the central axis Δ, thus decreases as a function of the distance to the central axis Δ. The thinning makes it possible to increase the amplitude of the vibrations propagating in the resonator, towards the sleeve.

In this configuration the piezoelectric transducers have a small thickness, typically of between 0.05 mm and 5 mm, preferably 0.5 mm for a radius r of 25 mm and 0.2 mm for a radius r of less than 10 mm, which maximizes the electric field, which can be of the order of 300 V/mm. This makes it possible to increase the mechanical stress, which is directly proportional to the electric field.

Each layer of piezoelectric material 13ₖ can be formed from a PZT (lead zirconate titanate) material, in particular product references PZ26, PZ27, PZ46 and PZ29 made by Ferroperm. Preferably, the coefficients d33 and d31, which reflect the coefficient of the deformation observed for an electric field applied (also perceived as a density of charges collected for a stress applied) are respectively:
at least 200 pC/N and preferably above 570 pC/N for the coefficient d33 quantifying the response of the piezoelectric material in a direction parallel to the direction of the electric field applied;
and at least 50 pC/N and preferably of the order of −240 pC/N for d31, which quantifies the response of the piezoelectric material in a direction perpendicular to the direction of the electric field applied.

The outer radius R of each resonator 10ₖ, defined about the central axis Δ, can extend up to 50 mm, or more. The flat portion of each resonator extends beyond a first radius R₁, smaller than the outer radius R defined above. Inside the first radius R₁, each resonator has a portion that becomes thinner in the direction of the central axis Δ. The first radius R₁ is for example equal to 50% of the outer radius R of the resonator.

Each resonator can extend along a thickness preferably less than 5 mm, for example 1 or 2 mm. The thickness is defined parallel to the longitudinal axis.

The resonator 10ₖ extends up to a cylindrical first sleeve 3ₖ that is coaxial with the central axis Δ. The diameter D of the sleeve 3ₖ is for example between approximately 0.2 mm and 2 mm and preferably close to 1 mm. The sleeve 3ₖ is preferably formed by an extension of the resonator 10ₖ, the resonator and the sleeve forming a monolithic part. The radius of the sleeve forms an inner radius of the first resonator 10ₖ. The height of each sleeve 3ₖ is preferably smaller than a wavelength of the waves propagating in the sleeve. The height of each sleeve 3ₖ is preferably equal to a half wavelength. The wavelength depends on the thickness of the sleeve and the resonance frequency. In practice, the height of the sleeve is less than 1 mm and preferably less than or equal to the thickness of the resonator in the portion applied against the piezoelectric transducer.

This makes it possible for the sleeve of each resonator to be as close as possible to the sleeve of another resonator, without being in direct contact. The spacing between two adjacent sleeves can be at least 0.05 mm.

Each resonator is formed from a deformable solid material, which can be a metal (titanium, stainless steel, aluminium or aluminium alloy, brass or another copper-based alloy, or a nickel-based alloy), an inorganic material (glass), an organic material (PEEK), or alumina.

The pump comprises a control unit 20, connected to each first electrode 12ₖ so as to apply a frequency-modulated polarization voltage to them. The modulation frequency depends on the dimension and on the material forming each resonator. The frequency is the same for each piezoelectric transducer. When the radius of each resonator is 25 mm, the modulation frequency can be approximately 25 kHz. When the radius of each resonator is 12.5 mm, the modulation frequency can be approximately 50 kHz. When the radius of each resonator is 6.5 mm, the modulation frequency can be approximately 100 kHz. In any event, the modulation frequency is preferably ultrasonic, so as to avoid the generation of an audible sound.

Each counter electrode 14ₖ is connected to a fixed potential, which can be common to all of the counter electrodes (for example an earth), or independent from the potential to which another electrode is connected, each independent potential being for example a floating earth.

Each resonator 10ₖ is configured to be deformed by a compression wave acting on each sleeve, so as to radially compress the capillary. Under the effect of the compression, the inner diameter of each sleeve oscillates according to a compressive radial resonance frequency. According to one option, each sleeve can be displaced parallel to the axis of the capillary according to a flexural radial resonance frequency.

More specifically, as the dimensions of the resonator are finite, a surface element situated on the inner wall defining each sleeve oscillates, describing an ellipse on each resonance period. The major axis s_{z} of the ellipse is parallel to the central axis Δ according to a flexural radial resonance frequency. A slight rolling effect is therefore imparted to the capillary. The resonator 10ₖ can vibrate at its fundamental frequency or at a harmonic frequency. In this latter case, the ratio between the major axis s_{z} and the minor axis sᵣ of the ellipse travelled along becomes closer to one. The movement becomes more circular for a higher harmonic frequency.

The resonators can be separated from each other by spacers 4. This makes it possible to stiffen the stack.

The thickness of the stack can be between 5 mm and 10 mm.

The transducers are offset along the longitudinal axis Δ, so that the compression moves along the central axis. The time offset is

$$\frac{2 π}{K} T ,$$

where T corresponds to the period in which each transducer was actuated. The resonance frequency is estimated to be at most 150 kHz for a fundamental frequency.

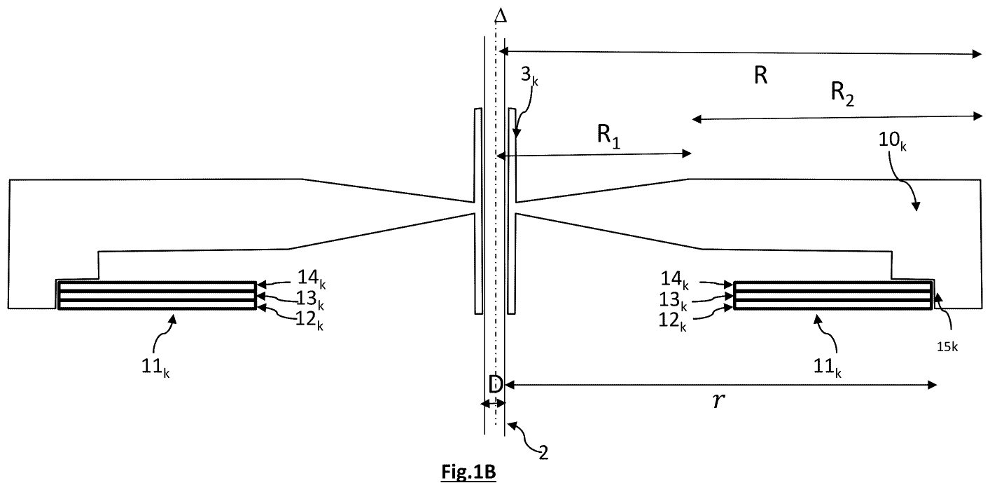

FIG. 2 illustrates a variant of the embodiment described with reference to FIGS. 1A and 1B, wherein the stack is accommodated in a housing. Each end of the capillary is conical, which facilitates a connection with a flexible capillary 5, for example made from silicone. In this example, the assembly formed by the resonators is held on a base 6ₐ to which a cover 6_{b} is clipped.

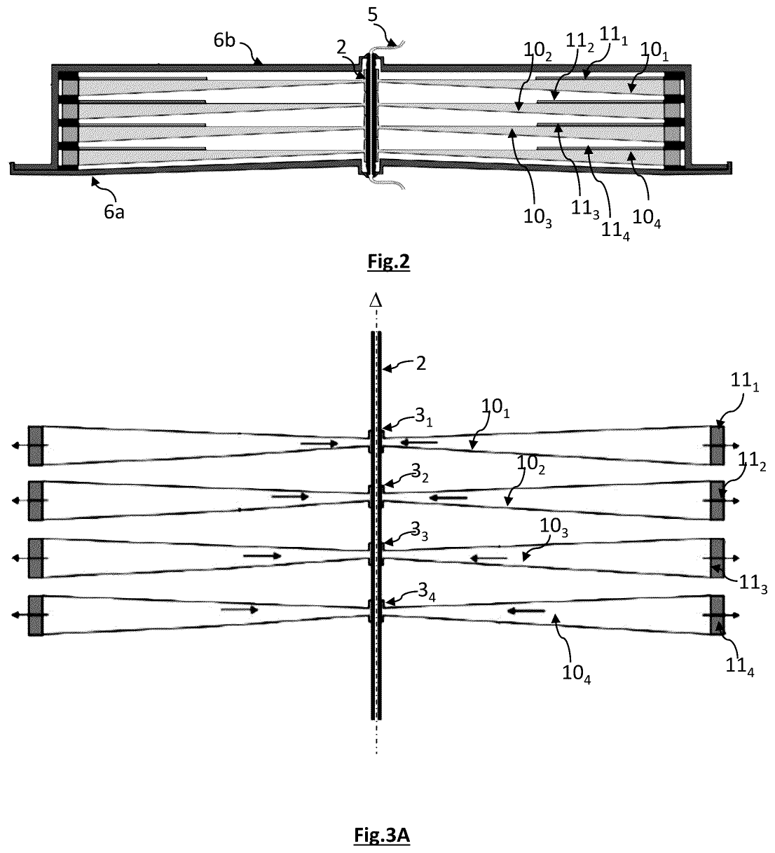

FIGS. 3A and 3B illustrate an embodiment similar to the embodiment in FIGS. 1A and 1B wherein each piezoelectric transducer is a ring arranged around the resonator to which it is coupled. Such a configuration is conducive to the generation of symmetric Lamb waves propagating radially and converging towards the central axis. This makes it possible to concentrate the compressive forces on the sleeve. In this case, the major axis of the ellipse travelled along by the displacement of a surface element of the sleeve at the interface with the capillary is oriented perpendicular to the central axis Δ of the capillary. The transducers are offset (or activated according to a time shift) along the central axis Δ, so that the compression associated with the major axis of the ellipse moves along the central axis. The time offset or shift is

$$\frac{2 π}{K} T ,$$

where T corresponds to the period in which each transducer was actuated.

The use of piezoelectric transducers arranged on the periphery of resonators makes it possible to increase the frequency, the resonators having a high thickness resonance frequency, in practice greater than a megahertz, or a lower frequency if a compressive radial resonance of the thin disc is utilized. This makes it possible to use a finer capillary, close to one tenth or a few tenths of a millimetre. Such a configuration is suitable for delivering low doses of active ingredient, or for an implanted pump, or for dispensing low volumes of fluids such as catalysts or costly products, or for slow and well-controlled supply of powders or various products (gas, lubricant, etc.) necessary for a particular production process or for maintenance.

FIG. 3B shows an annular piezoelectric transducer: the electrode 12ₖ and the counter electrode 14ₖ are annular, as is the piezoelectric layer 13ₖ. In this example, the thickness of the piezoelectric layer 13ₖ is 1 mm, the diameter of the electrode 12ₖ being 22 mm for example. In order to ensure electrical isolation between the electrode 12ₖ and the resonator 10ₖ to which it is attached, the piezoelectric layer 13ₖ can be bevelled (bevel not shown) over a depth of at least 0.1 mm on the edges of the outer electrode 12ₖ. In the radial direction, the width of the piezoelectric layer can be between 0.5 mm and 1 mm. The resonance frequency for a thickness of 1 mm is approximately 2 MHz.

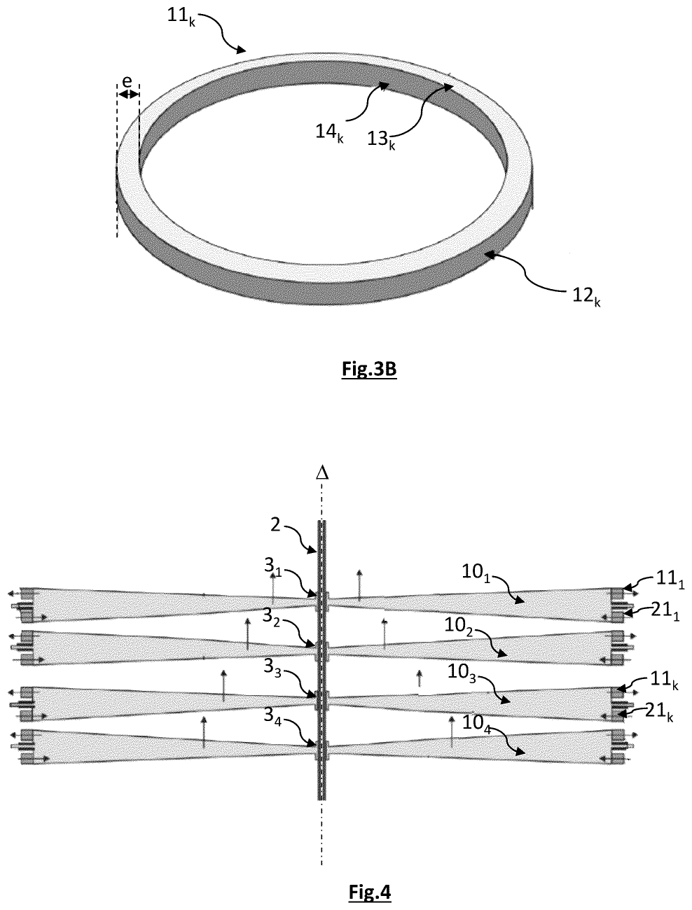

FIG. 4 shows a similar configuration to the one described with reference to FIGS. 3A and 3B. In this configuration, each resonator is coupled to two annular piezoelectric transducers 11₁, 21₁, activated in phase opposition and arranged around the resonator. Such a configuration is conducive to the generation of antisymmetric Lamb waves propagating radially, converging in the thin region causing a shearing of the capillary 2, the shear propagating along a predetermined direction. In this case, the major axis s_{z} of the ellipse travelled along by a surface element at the interface between the sleeve and the capillary is parallel to the central axis Δ of the capillary.

As in the preceding embodiment, the transducers coupled to two adjacent resonators are offset along the longitudinal axis Δ, so as to propagate the shear stress gradually along the capillary. The time offset (or shift) is

$$\frac{2 π}{K} T ,$$

where T corresponds to the period in which each transducer was actuated. During the same phase, the first transducer 11ₖ of a resonator 10ₖ is activated, so as to induce a bowing of the resonator in one direction in its central region, while the second transducer 21ₖ of a resonator 10ₖ is activated, so as to induce a bowing of the resonator in the same direction, and must therefore be offset by π, on the central axis. This is obtained if the outer electrodes of the transducers 11ₖ and 21ₖ (respectively) are subjected to the same electric excitation voltage and if the electric dipole moments of the piezoelectric materials of the transducers 11ₖ and 21ₖ are of opposite directions as indicated by the arrows passing through the transducers shown in FIG. 4.

Pumping by peristaltic shear effect is thus obtained, due to the progressive shearing of the capillary 2, which causes a displacement of the fluid by viscosity and incompressibility of the fluid along the central axis. The efficiency of pumping is increased by a displacement component sᵣ in the plane of each resonator, corresponding to the minor axis of the displacement ellipse and resulting in a compression of the capillary tube. The symmetric and antisymmetric acoustic modes S0 and A0 initiated at the periphery of the resonators in FIGS. 3A and 4 both comprise components of compression displacement sᵣ and shear s_{z} of the capillary corresponding to the major axis and the minor axis of the ellipse along which a displacement vector travels, but with a dominant compression component in sᵣ for the symmetric mode and a dominant shear component in s_{z} for the antisymmetric mode. These two vibration modes are considered to be peristaltic.

The configuration shown in FIG. 4 also makes it possible to form symmetric acoustic modes S0, as shown schematically in FIG. 3A, when the outer electrodes of the two piezoelectric transducers 11ₖ, 21ₖ connected to each resonator 10ₖ are in phase opposition and if the electric dipole moments of the piezoelectric transducers of the two piezoelectric transducers 11ₖ, 21ₖ are of opposite directions as shown in FIG. 4.

When the dipole moments of the transducers 11ₖ and 21ₖ are oriented in the same direction, and the excitation voltages are in phase opposition, an antisymmetric vibration mode A0 is obtained as described above. When the excitation voltages are in phase, a symmetric vibration mode S0 is obtained.

A symmetric vibration mode has the effect of compressing the capillary and producing a displacement of the capillary/sleeve interface travelling along an ellipse with a major axis orthogonal to the axis Δ. This vibration is transmitted into the wall of the capillary in the form of a flexural wave having axial symmetry relative to the axis Δ.

An antisymmetric vibration mode has the effect of producing a displacement of the capillary/sleeve interface wherein the major axis of the ellipse is parallel to the axis Δ, which results in a shearing of the capillary in accordance with the axial symmetry relative to the axis Δ.

As a result, if the electrical phase shift between the two transducers 11ₖ and 21ₖ is varied from 0 to 180°, there is a continuous switch from predominant generation of a symmetric deformation mode to predominant generation of an antisymmetric mode relative to the mid-plane of the resonator 10ₖ. The two symmetric and antisymmetric vibration modes are created simultaneously but in different proportions depending on the phase shift and at different phase speeds that are higher for the symmetric mode than for the antisymmetric mode. Given that these vibration modes can coexist simultaneously and elastically in the resonator, a specific electric excitation phase shift of between 0 and 180° C. must simply be imposed in order to obtain a vibration of the wall of the capillary so that each surface element of the capillary moves along an ellipse, the major axis of which can vary from an orientation perpendicular to the axis Δ to an orientation parallel to the axis Δ as a function of the phase shift that has been selected between the two transducers 11ₖ and 21ₖ.

Whatever the configuration, the peak-to-peak polarization voltage can vary for example from a few volts to several hundred volts, the voltage acting on the amplitude of the out-of-plane deformation component of the resonator. The amplitude of this deformation component acts on the volume of fluid at the inner interface of the capillary undergoing the elliptical rolling effect that drives it, and therefore on the pumping pressure.

1. A Peristaltic pump, configured to pump a liquid along a capillary, the pumping resulting from a stress exerted on the capillary, successively along a pumping direction, the peristaltic pump comprising:
K annular piezoelectric transducers, extending about a central axis, each piezoelectric transducer being configured to be polarized by a polarization electrode, K being an integer greater than or equal to 2;
K resonators, formed from a deformable solid material, each resonator being connected to a piezoelectric transducer, and extending about the central axis, becoming thinner towards the central axis, each resonator being configured to deform under the effect of the polarization of the piezoelectric transducer to which it is connected;
a control unit, configured to polarize each polarization electrode according to a polarization voltage, modulated according to a modulation frequency;
wherein:
each resonator is connected to a sleeve, extending about the central axis;
each resonator is arranged so that the sleeves of each resonator are aligned along a central axis;
the control unit is configured to polarize each resonator successively, as a function of their respective ranks, so that under the effect of the polarization, each resonator is deformed successively, the deformation propagating up to the sleeve, and resulting in the successive deformation of the capillary arranged between the sleeves, about the central axis.

2. The pump according to claim 1, wherein the stress is a compression and/or a displacement along the central axis.

3. The pump according to claim 1, wherein the modulation frequency is greater than 20 kHz.

4. The pump according to claim 1, wherein:
each resonator comprises a peripheral portion, delimited by two flat faces, and a central portion, extending between the peripheral portion and the sleeve, the central portion becoming thinner towards the sleeve;
each piezoelectric transducer extends along one of said flat faces.

5. The pump according to claim 4, wherein the flat face along which each piezoelectric transducer extends is delimited by a shoulder, the piezoelectric transducer being force-fitted against the shoulder, so that the shoulder encircles the piezoelectric transducer.

6. The pump according to claim 1, wherein each resonator is coupled to at least one annular piezoelectric transducer, the annular piezoelectric transducer extending around the resonator, so as to cause a radial compression of the resonator, in the direction of the central axis.

7. The pump according to claim 6, wherein each piezoelectric transducer is force-fitted around the resonator to which it is connected.

8. The pump according to claim 1, wherein each resonator is coupled to two annular piezoelectric transducers, extending around the resonator, and offset along a direction parallel to the central axis, wherein the central unit is configured to power the piezoelectric transducers so as to cause a displacement of the sleeve along the central axis and/or a compression of the sleeve perpendicular to the central axis.

9. The pump according to claim 8, wherein
each piezoelectric transducer having a dipole moment, the dipole moments of the piezoelectric transducers coupled to the same resonator are oriented in the same direction;
the control unit is configured to power said piezoelectric transducers in phase;
so as to cause a radial compression of the sleeve that is predominant over a displacement of the sleeve along the central axis.

10. The pump according to claim 8, wherein,
each piezoelectric transducer having a dipole moment, the dipole moments of the piezoelectric transducers coupled to the same resonator are oriented in two opposite directions
the control unit is configured to power said piezoelectric transducers in phase opposition;
so as to cause a radial compression of the sleeve that is predominant over a displacement of the sleeve along the central axis.

11. The pump according to claim 8, wherein
each piezoelectric transducer having a dipole moment, the dipole moments of the piezoelectric transducers coupled to the same resonator are oriented in the same direction;
the control unit is configured to power said piezoelectric transducers in phase opposition;
so as to cause a displacement of the sleeve along the central axis that is predominant over a compression of the sleeve.

12. The pump according to claim 8, wherein, each piezoelectric transducer having a dipole moment:
the dipole moments of the piezoelectric transducers coupled to the same resonator are oriented in two opposite directions;
the control unit is configured to power said piezoelectric transducers in phase;
so as to cause a displacement of the sleeve along the central axis that is predominant over a compression of the sleeve.

13. The pump according to claim 8, wherein the control unit is configured to power said piezoelectric transducers according to a predetermined phase shift, of between 0° and 180°, so as to obtain a predetermined ratio between the displacement of the sleeve along the central axis and the compression of the sleeve.

14. The pump according to claim 1, comprising the capillary, the capillary being force-fitted between each sleeve.

15. The pump according to claim 14, wherein the capillary is rigid.
